SEQUENCE LISTING (4 sequences)

SEQ ID NO: 1 - DNA, 51 bp, synthetic construct
  source 1..51: mol_type = other DNA
cagcaccgaccttgtgctttgggagtgctggtccaagggcgttaatggaca

SEQ ID NO: 2 - DNA, 26 bp, synthetic construct
  source 1..26: mol_type = other DNA
ggacattttttttttttttttcagca

SEQ ID NO: 3 - DNA, 67 bp, synthetic construct
  source 1..67: mol_type = other DNA
aaaaaaaatgtccattaacgcccttggaccagcactcccaaagcacaaggtcggtgctgaaaaaaaa

SEQ ID NO: 4 - DNA, 16 bp, synthetic construct
  source 1..16: mol_type = other DNA
aaaaaaaaaaaaaaaa

(19) United States
(12) Patent Application Publication
(10) Pub. No.: US 2023/0295751 A1
(43) Pub. Date: Sep. 21, 2023

(54) Biological Detection Sensor Chip and COVID-19 Test Kit

(71) Applicant: Xudong Cao, Ottawa, Ontario (CA)

(72) Inventors: Xingkai Hao, Ottawa (CA); Xudong Cao, Ottawa (CA)

(21) Appl. No.: 18/185,344

(22) Filed: Mar. 16, 2023

Related U.S. Application Data
(60) Provisional application No. 63/269,610, filed on Mar. 18, 2022.

(51) Int. Cl. C12Q 1/70; C12Q 1/682; B01L 3/00

(52) U.S. Cl. CPC C12Q 1/701; C12Q 1/682; B01L 3/502761; C12Q 1/6804

(57) ABSTRACT

The invention discloses a biosensor chip and a COVID-19 test kit. The COVID-19 test kit includes a biosensor chip, rolling circle amplification (RCA) primers, RCA padlock probes, and ssDNA conjugated nanoparticles (i.e., ssDNA-NPs probe). In this invention, the surface of the biosensor chip is modified with functional polymers; subsequently, detection ligands are grafted on the modified sensor chip surface to capture the targets. The developed biosensor chip achieves rapid and ultra-sensitive on-site detection of COVID-19 infections. In addition, a signal amplification method is employed using RCA products hybridized ssDNA-NPs probes (i.e., RCA-NPs) complex to amplify the detection signal and to improve the detection sensitivity. The RCA sequence in this invention is designed to generate tandem repeating aptamers and ssDNA-NPs probe hybridization sites so that multiple ssDNA-NPs probes can be hybridized with the RCA products and attached to the captured targets in order to increase the target mass, resulting in detection signal intensification.

This disclosure contains a sequence listing file, submitted in XML file format, named “Sequence-Listing.xml” and created on Mar. 16, 2023, with 5 kilobytes in size. The material in the above-identified XML file is incorporated herein by reference in its entirety.

### TECHNICAL FIELD

This invention relates to the field of biotechnology, in particular to a biological detection sensor chip and a COVID-19 test kit.

### BACKGROUND

The current COVID-19 pandemic has caused millions of deaths and affected economies around the world. Currently, diagnostic tests that are both rapid and sensitive for COVID-19 infections are limited. The primary method to diagnose COVID-19 is nucleic acid based testing by polymerase chain reaction (PCR). Although this method is sensitive, it requires complex nucleic acid extraction, a long time for nucleic acid amplification, and highly trained personnel to collect and handle the samples. As a result, it takes up to several days to get the results. Another method to detect COVID-19 patients is to use rapid test strips; however, the sensitivity of paper strips is generally low, which can only effectively detect patients with high viral loads, while patients with low viral loads often show false negative test results. Therefore, the rapid test strip is not an effective method to diagnose COVID-19 infections.

Biosensors are composed of a biosensor chip that captures target signal transduction components that transduce the capture events into readable signals. Biosensors have the advantages of rapid detection, portability, convenience, and low cost, and they can be used for both quantitative and semi-quantitative analysis of the analytes. Therefore, biosensors are ideal methods for the rapid detection of COVID-19 infections. Several biosensors have been developed to detect COVID-19-related biological targets, including nucleic acids, antibodies, and proteins. However, these biosensors are not sensitive enough, and therefore they cannot meet the needs for COVID-19 infection diagnosis. The main reason for the low sensitivity issue is the followings: First, the number of capturing ligands (i.e., antibodies and aptamers) on the detection surface of the biosensor chip is relatively low to allow effective capturing of targets. This is particularly true when the concentration of the target analytes in the sample is low, leading to low detection sensitivities. At present, there is no effective method to improve the density of capturing ligands on the surface of biosensor chips. Second, there are nonspecific interactions between the capturing surface biosensor chip and the sample matrix, resulting in high nonspecific background noises, leading to a low signal-to-noise ratio and thus reducing the detection sensitivity of the biosensor. To solve this issue, a reference signal is typically collected to compensate for the nonspecific background noise. For example, a two-channel design is usually used for micro fluidic-based localized surface plasmon resonance (LSPR) biosensors. Specifically, one channel is used to collect overall detection signals, and the other is employed for collecting nonspecific background noise as a reference. However, this approach complicates the channel designs and requires more sophisticated fluidic control and optical systems. Another way to control nonspecific adsorption is to use blocking reagents (such as bovine serum albumin (BSA)) to reduce the nonspecific interactions with the biosensor chip surface. However, blocking reagents may also shield target-capturing ligands, cross react with other components in sample matrices, and damage the structures of the targets, thus ultimately affecting detection sensitivities and complicating sample preparations. Finally, since the issue of the surface nonspecific adsorption of the sensor chip has not been effectively solved, any signal amplifications would also likely increase the background noises, making the signal amplification less effective in enhancing the signal-to-noise ratio.

Therefore, improving the detection performance of existing biosensor chips is an urgent problem to be solved.

### SUMMARY OF THE INVENTION

The invention aims to overcome the deficiencies of the prior art, provide a novel biosensor chip design, and improve the detection performance of the biosensor chip. To realize the above objectives, the invention employs the following technical scheme:

The invention provides a biosensor chip, including a sensor chip body whose detection surface is in a form of a template layer; the template layer has multiple binding sites that bind to the detection ligands.

The invention provides a template layer on the detection surface of the sensor chip body, and the template layer has multiple binding sites that bind to the detection ligands. The template layer can provide multiple binding sites to conjugate multiple detection ligands, which improves the number of detection ligands on the surface of the biosensor chip, and thus improving the target-capturing efficiency.

The biosensor chip of the invention can be different types of sensor chips, such as surface plasmon resonance biosensor chip (SPR), local surface plasmon resonance biosensor chip (LSPR), electrochemical chip, surface-enhanced Raman chip, or other types of sensor chips.

In addition, the detection surface of the chip body can also be different structural forms, such as nanoparticle-, coating-, nanowell-based structural forms, or other structural forms.

The detection ligands in this invention can be aptamers, antibodies, peptides, receptors, polymers, enzymes, or others. In a preferred embodiment, the detection ligands are aptamers.

The template layer is functional polymers immobilized on the detection surface of the chip body. Functional polymers (such as dendrimers, linear polymers, and crosslinked polymers) have been increasingly used to improve the performance of detection surfaces. Functional polymers can be used as an excellent template to decorate the detection surfaces with multiple binding sites that can further conjugate with a large number of capturing ligands (e.g., aptamers and antibodies) on the detection surfaces to improve the target capturing efficiency. In addition, the detection surfaces modified with functional polymers are rendered excellent non-fouling properties, which can reduce nonspecific interactions and improve the detection signal-to-noise ratio.

In the invention, the functional polymers can be immobilized on the detection surfaces of the biosensor chip body by either physical adsorption, chemical binding, or other methods to form a template layer. For example, a biosensor chip comprises a substrate, metallic materials, and functional polymers. The substrate material can be glass, poly(methyl methacrylate) (PMMA), cycloolefin, poly(styrene), polymer coatings, or other materials. The metallic materials are immobilized on the surface of the substrate to provide signal transduction functions; the surface metallic materials can be gold, silver, platinum, copper and other metallic materials, or metal-covered non-metallic materials. The functional polymers are overlaid on top of the surfaces of the substrate surface, the metallic materials, or any other surface of exposed areas (i.e., the functional polymers can cover the entire detection surface of the chip body to prevent nonspecific adsorptions).

The functional polymers as a template layer have the following functions:
(1) The functional polymers immobilized on the detection surface render the resulting surface nonfouling performance, which prevents non-targets from interacting with the sensor chip surface, thus reducing nonspecific background noise thereby improving the detection signal-to-noise ratio;
(2) The functional polymers can be used as templates, and when immobilized on the detection surface, they can provide multiple binding sites to conjugate multiple copies of detection ligands in order to increase the number and density of detection ligands on the surface of the biosensor chip, thus improving the target capturing efficiency.

In addition, the molecular structures of the functional polymers in the invention can also be in various shapes. In a preferred embodiment, the molecular structures of the functional polymers include at least one of the dendritic, linear, and crosslinked molecules.

In other words, the functional polymers can be dendritic, linear, crosslinked molecules, or any combination of the above, depending on the surface characteristics of the substrates and surface metallic materials of the biosensor chip body.

In a preferred embodiment, the functional polymer is PAMAM dendrimer; the PAMAM dendrimers comprise at least one of Generation 3.5 carboxylated PAMAM dendrimers (i.e., G3.5-COOH) and the Generation 4 aminated PAMAM dendrimers (i.e., G4-NH₂).

For example, the detection surface of the chip body can be successively modified with G3.5-COOH and G4-NH₂ to make the surface nonfouling.

Subsequently, the immobilized G4-NH₂ acts as a template layer to provide multiple binding sites to conjugate with many detection ligands to form the biosensor chip.

As an advanced amplification technique, rolling circle amplification (RCA) reactions can generate long single-strand DNA sequences with hundreds or thousands of tandem repeating units—based on a circular template—that can be designed to either capture specific targets, amplify signals, or do both. As a result of its inherent advantages, RCA has been widely used in biosensors to amplify detection signals. For example, Jiang et al. applied the RCA with fluorescence probes to achieve in-situ signal amplification of E. coli detection on a microfluidic device, and the result showed that the detection signals were enhanced by up to 50 times. However, the RCA method for improving the signal of COVID-19 detection in biosensors has never been developed. Therefore, the invention is necessary, as it develops an RCA sequence to improve the detection signals of the SARS-CoV-2 virus to enhance the detection sensitivity of the biosensor chip to COVID-19 infections.

Therefore, the invention also provides a COVID-19 test kit that includes the above-mentioned biosensor chips, RCA primers, RCA padlock probes, and ssDNA-NPs probes.

The invention applies the above-mentioned biosensor chips to detect the SARS-CoV-2 virus.

In the invention, the aptamer can specifically capture the receptor binding domain of the spike protein (SRBD) on the surface of the SARS-COV-2 virus, and the sequence is: SEQ ID NO: 1.

To improve the detection sensitivity, the invention develops a signal amplification method by hybridizing the rolling circle amplification product with nanoparticle probes (RCA-NPs) to amplify the detection signal and improve the detection sensitivity.

In the invention, the RCA reaction produces RCA products composed of series repeating aptamers and ssDNA-NPs probe hybridization sites; ssDNA conjugates with NPs to form ssDNA-NPs probes; ssDNA-NPs probes hybridize with RCA products to form RCA-NPs complexes; the RCA-NPs complex can bind to the captured virus through the generated repeating aptamers.

In other words, the rolling circle amplified sequence in the invention can generate repeating series of aptamers and ssDNA-NPs probe hybridization sites, so that many ssDNA-NPs probes can hybridize with the RCA product and attach to the captured virus through a large number of RCA-generated aptamers, thus increasing the target mass and enhancing the detection signal.

In a preferred embodiment, the NPs comprise at least one of the gold NPs (AuNPs), silver NPs, platinum NPs, copper NPs, and non-metallic NPs with metal coatings.

In a preferred embodiment, the sequence of the RCA primer is: SEQ ID NO:2.

In a preferred embodiment, the sequence of the RCA padlock probes is: SEQ ID NO:3.

In a preferred embodiment, the sequence of the ssDNA is: SEQ ID NO:4.

The benefits of the invention compared with prior arts are:
(1) The invention uses functional polymers to modify the surface of the biosensor chip, and subsequently grafts detection ligands on the modified surface to realize rapid and ultra-sensitive on-site detection of analytes. The design has the following unique features: a) the surface-immobilized functional polymers have excellent nonfouling properties, preventing non-targets from interacting with the sensor surface, reducing nonspecific background noises and thus improving detection signal-to-noise ratio; b) the immobilized functional polymers act as templates to provide multiple binding sites for conjugating multiple copies of detection ligands, which improves the number and density of detection ligands on the surface of biosensor chips, thus improving the target capturing efficiency;
(2) The invention employs a signal amplification method using RCA-NPs to amplify the detection signal and improve detection sensitivity. The rolling circle amplified sequence in the invention can generate tandem repeating units of both aptamers and ssDNA-NPs probe hybridization sites, so that many ssDNA-NPs probes can hybridize with the RCA product and attach to the captured virus through a large number of generated aptamers, thus increasing the target mass and enhancing the detection signal.

### BRIEF DESCRIPTION OF THE DRAWINGS

In order to better illustrate the technical solutions of the embodiments of the present invention, the descriptions of drawings to illustrate listed embodiments are briefly listed. It should be clear that the drawings in the following description are some embodiments of the present invention, and it is obvious for those skilled in the art to obtain other drawings based on these drawings without creative efforts.

FIG. 1 is the different sensor chip surfaces modified with different functional molecules followed by conjugated with detection ligands;

FIG. 2 is the schematic illustration showing surface-modified sensor chips for the detection of the SARS-CoV-2 virus and signal amplification using RCA-NPs;

FIG. 3 is the schematic illustration showing the general approaches of surface modification of the LSPR sensor chip and target detection described in Embodiment 2 of the invention;

FIG. 4 shows the fluorescence intensity changes before and after surfaces modified with fluorescence-labeled molecules described in Embodiment 2 of the invention) is the fluorescence intensities of original sensor chip surfaces modified by FITC labeled G3.5-COOH molecules (i.e., FITC-G3.5) to confirm the success of G3.5 immobilization; B) is the fluorescence intensities of G3.5-COOH modified surfaces modified by rhodamine-labeled G4-NH₂ molecules (i.e., rhodamine-G4) to confirm the success of G4 immobilization; C) is the fluorescence intensities of (G3.5+G4) modified surfaces conjugated with cy3-labeled aptamers (i.e., cy3-aptamer) to confirm the success of aptamer conjugation. Error bars indicate standard deviation, n=3;

FIG. 5 is the comparison of LSPR signals of each step of surface modifications described in Embodiment 2 of the invention;

FIG. 6 shows the ultraviolet-visible spectral changes between ssDNA, AuNPs, and ssDNA-AuNPs described in Embodiment 2 of the invention;

FIG. 7 is a gel electrophoresis image of the RCA product described in Embodiment 2 of the invention;

FIG. 8 shows LSPR sensor graphs of different tests to evaluate performances of different sensor chip surface modifications described in Embodiment 2 of the present invention) is the detection background noises of different sensor chips as evaluated in a nonspecific adsorption experiment using BSA (1 mg/mL). B) is the comparison of the amount of aptamers immobilized on different sensor chips. C) is the detection signals of SRBD (377.36 nM) tested on different modified surfaces. Legends show surfaces with different modification strategies, including gold-aptamer (curve \textcircled{1}), G3.5-aptamer (curve \textcircled{2}), G4-aptamer (curve \textcircled{3}), and (G3.5+G4)-aptamer (curve \textcircled{4}).

FIG. 9 compares the detection performance between the gold-aptamer and (G3.5+G4)-aptamer modified sensor chips for SRBD detection escribed in Embodiment 2 of the present invention;

FIG. 10 is the LSPR sensor graph for detecting the SARS-CoV-2 virus using (G3.5+G4)-aptamer modified LSPR sensor chip with RCA-AuNPs signal amplification; the inset shows the relationship between the signals and the target concentrations; error bars indicate standard deviations, n=3; and

FIG. 11 is the detection specificity and influence of sample matrices. A) The detection specificity of the (G3.5+G4)-aptamer modified LSPR sensor chip. B) Influence of sample matrices on detection performances.

### DETAILED DESCRIPTION

The technical solutions in the embodiments of the present invention are clearly described below with reference to the drawings in the embodiments of the present invention. It should be noted that the described embodiments are some, not all, embodiments of the present invention. All other embodiments that can be derived by a person skilled in the art from the embodiments given herein without making any creative effort shall fall within the protection scope of the present invention.

It is understood that the terminology used in the description of the invention herein is for the purpose of describing particular embodiments only and is not intended to be limiting of the invention. As used in the specification of the present invention and the appended claims, the singular forms “a,” “an,” and “the” are intended to include the plural forms as well, unless the context clearly indicates otherwise.

It is also to be understood that the term “and/or” as used in this specification and the appended claims refers to and includes any and all possible combinations of one or more of the associated listed items.

### Embodiment 1

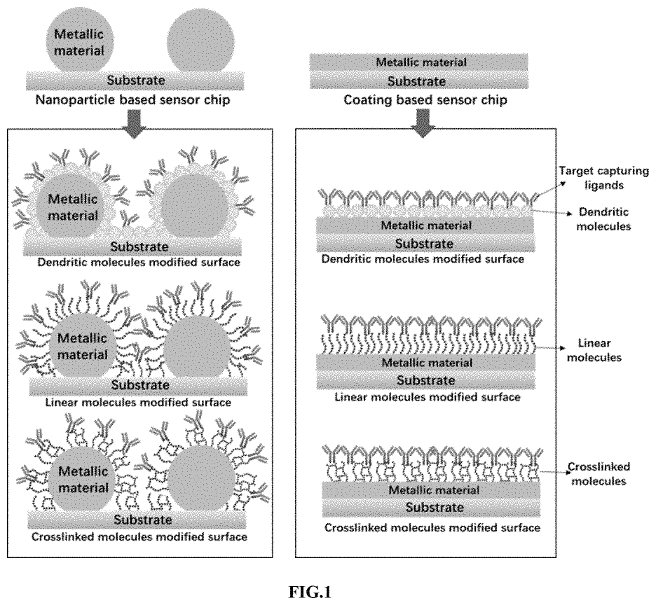

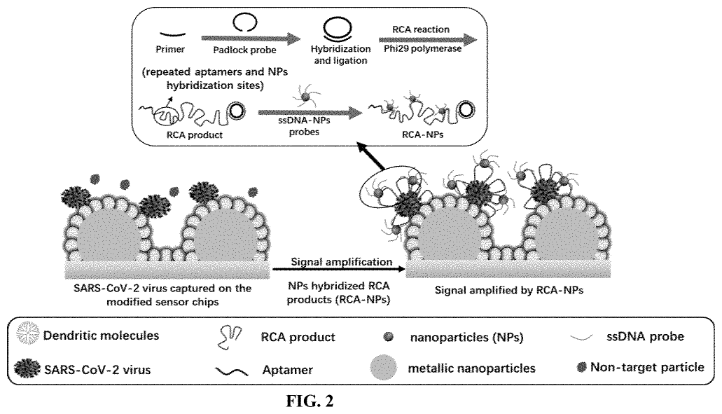

As shown in FIG. 1, the present embodiment is a biosensor detection chip, including a sensor chip body whose detection surface is in a form of a template layer; the template layer has multiple binding sites that bind to the detection ligands. The template layer is formed by immobilizing functional polymers on the detection surface of the chip body. Moreover, the molecular structures of the functional polymers in this embodiment may also be in a variety of shapes. For example, in the present embodiment, the functional polymer material includes at least one of the dendritic, linear, and crosslinked polymers. In other words, in this embodiment, the functional polymers can be dendritic, linear, crosslinked molecules, or any combination of the above, depending on the surface characteristics of the substrates and surface metallic materials of the biosensor chip body.

In the present embodiment, the detection surface of the chip body is featured with a template layer that provides multiple binding sites to conjugate with multiple copies of detection ligands, which improves the number and density of detection ligands on the detection surface of the biosensor chip and thus improves the target capturing probability.

In the present embodiment, the biosensor chip can be of different types of sensor chips, such as surface plasmon resonance biosensor chip (SPR), local surface plasmon resonance biosensor chip (LSPR), electrochemical chip, surface-enhanced Raman chip, or other types of sensor chips.

In addition, the detection surface of the chip body can also be different structural forms, such as nanoparticle-, coating-, nanowell-based structural forms, or other structural forms.

In the present embodiment, the surface-conjugated detection ligands can be aptamers, antibodies, peptides, receptors, polymers, enzymes, or others.

In the present embodiment, the functional polymers can be immobilized on the detection surface of the chip body by either physical adsorption, chemical binding, or other methods to form a template layer. For example, a biosensor chip can comprise a substrate, metallic materials, and functional polymers. The substrate is used to allow the metallic materials and functional polymers to be immobilized on its surface; the substrate materials can be glass, poly(methyl methacrylate) (PMMA), cycloolefin, poly(styrene), polymer coatings, or other materials. The metallic materials are immobilized on the substrate surface to provide signal transduction functions; the metallic materials can be gold, silver, platinum, copper, and other metallic materials, or metal-covered non-metallic materials. The functional polymers are overlaid on top of the surfaces of the substrate surface, the metallic materials, or any other surface of exposed areas (i.e., the functional polymers can cover the entire detection surface of the chip body to prevent nonspecific adsorptions).

In the present embodiment, functional polymers acting as the template layer have the following functions:
(1) Functional polymers immobilized on the detection surface have excellent nonfouling performance, which prevents non-targets from interacting with the sensor chip surface to reduce nonspecific signals and thus improves the detection signal-to-noise ratio;
(2) The surface immobilized functional polymers can be used as templates to provide multiple binding sites to conjugate multi-copy detection ligands, which improves the number and density of detection ligands on the surface of the biosensor chip and thus improving the target capturing efficiency.

### Embodiment 2

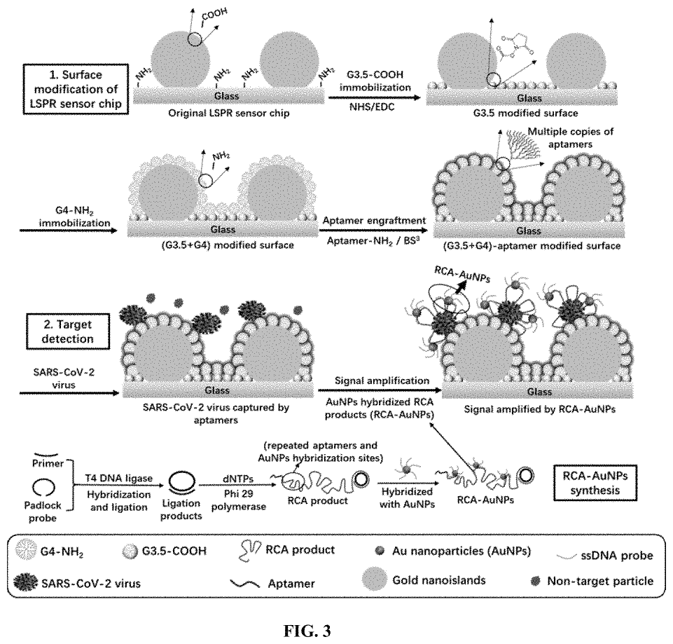

In the current embodiment, a commonly used local surface plasmon resonance (LSPR) sensor chip is used as an example to illustrate the superiority of the surface modification approach and signal amplification method. The surface modification of the LSPR sensor chip and detection process is shown in FIG. 3. Specific steps include:

1. Since the exposed glass areas of the original LSPR sensor chip presented —NH₂ functional groups while the gold nanoislands presented —COOH groups, the sensor chip surface was treated with a G3.5-COOH immobilization solution, including 50 μM G3.5-COOH, 0.1 M N-hydroxysuccinimide (NHS) and 0.1 M N-ethyl-N′-(3-(dimethylamino)propyl) carbodiimide (EDC) in PBS (pH 7.4), for 10 min to immobilize G3.5-COOH on the glass areas and to convert the carboxyl surface functional groups on both the glass areas and the gold nanoislands into NHS esters.

2. The sensor chip obtained in Step 1 was immediately soaked into a 100 μM G4-NH₂ PBS (pH 7.4) solution for 10 min. In this modification step, the G4-NH₂ molecules were immobilized onto both the gold nanoislands and the immobilized G3.5 molecules. The immobilized G4-NH₂ molecules would be used as multi-handled templates to provide multiple binding sites (i.e., —NH₂ groups) in the next step to further conjugate aptamers.

3. The sensor chip obtained in Step 2 was soaked into an aptamer-conjugation solution, including 200 μM aptamer-NH₂ and 20 mM bis(sulfosuccinimidyl)suberate (BS3) in PBS (pH 7.4), for 20 min to conjugate aptamers onto the G4 templated surface. Subsequently, an ethanolamine-HCL solution (1 M, pH 8.5) was employed to treat the resulting surface for 5 min to deactivate the reaction.

As can be seen from above, the present embodiment sequentially modified the LSPR sensor chip surface with G3.5-COOH and G4-NH₂ to render the resulting surfaces nonfouling. The immobilized G4 dendrimers acted as multi-handled templates to allow for subsequent conjugation of numerous copies of aptamers specific for binding the SARS-CoV-2 virus.

4. Specific Steps for Target Detection and Signal Amplification

In the present embodiment, the modified sensor chip was used to capture the SARS-CoV-2 virus. The captured viral particles were then conjugated with RCA products hybridized ssDNA-AuNPs probes (i.e., RCA-AuNPs) to enhance the detection signals by increasing the target mass and plasma coupling effects. Specifically, the LSPR sensor chip obtained in Step 3 was loaded into a microfluidic-based LSPR biosensor. Subsequently, samples containing the SARS-CoV-2 virus were injected into the sensor chip at a flow rate of 30 μL/min for 3.3 minutes to enable the surface aptamers to capture the viral particles. After this step, RCA-AuNPs was injected into the sensor chip at a flow rate of 30 μL/min for 3.3 min to interact with the surface-captured virus particles to amplify detection signals. After the detection, the captured SARS-CoV-2 virus could be released by injecting 10 mM glycine-HCL (pH 2.0) solution at a flow rate of 150 μL/min for 0.67 min to achieve sensor chip regeneration.

The method for preparing the above-mentioned RCA-AuNPs is as follows: The preparation of RCA-AuNPs consists of three parts: (1) synthesis of ssDNA-AuNPs nanoprobes; (2) synthesis of RCA products; and (3) synthesize RCA-AuNPs.

(1) Synthesis of ssDNA-AuNPs Nanoprobes

In the present embodiment, 6 μL of disulphide functionalized ssDNA (1 mM) (SEQ ID NO:4, Table 1) were deprotected using 14 μL of freshly prepared TCEP-HCl solution (17.14 mM) at room temperature for 2 h, after which the resulting ssDNA solution was transferred into a conjugation solution prepared by mixing 30 μL of PB buffer (1 M, pH=8.0, containing 1% (w/w) SDS) with 2950 μL of citrate-stabilized AuNPs (particle diameters can be 10-100 nm, concentration is around 3.8×10¹⁰ particles/mL) and allowed to react for 1 h. Subsequently, a salt-aging solution containing 2M NaCl, 0.01 M PB buffer (pH 8.0), and 0.01% (w/w) SDS was slowly added into the conjugation solution to allow 0.1 M of NaCl increment, followed by 10 s sonication and 20 min incubation at room temperature. This process was repeated 7 times until the final concentration of NaCl reached 0.7 M, after which the resulting solution was incubated at 4° C. for 16 h. Finally, to remove the unreacted reagents, the resulting solution was centrifuged for 20 min at 400-22,000×g (depending on the size of AuNPs), and the supernatant was removed, leaving the ssDNA-AuNPs conjugates at the bottom. The ssDNA-AuNPs were then resuspended in PBS (0.01 M, pH 7.4). This washing step was repeated for a total of three supernatant removals.

(2) Synthesis of RCA Products

In the present embodiment, circular templates for RCA were firstly prepared: 1 μL 100 μM RCA primers (100 μM) (SEQ ID NO:2, Table 1) and 1 μL of padlock probes (100 μM) (SEQ ID NO:3, Table 1) were mixed with 86 μL of nuclease-free water. Subsequently, The reaction mixture was treated at 95° C. for 10 min, quickly cooled in an ice bath for 1 min, and incubated at 37° C. for 30 min to allow hybridization between primers and padlock probes. After this step, the resulting solution was spiked with 10 μL of T4 ligation buffer (10×) and 2 μL of T4 DNA ligase (5 U/μL) to react at room temperature for 2 h in order to ligate the padlock probes, after which the reaction was terminated by heating at 65° C. for 10 min to obtain the final products of circular templates. This step was followed by an RCA reaction: the obtained circular template solution was mixed with 10 μL of phi29 DNA polymerase (10 U/μL), 40 μL of dNTP (10 mM), 100 μL of phi29 polymerase buffer (10×), and 750 μL of nuclease-free water. This reaction mixture was allowed to react at 37° C. for 1 h and subsequently heated at 65° C. for 10 min to stop the reaction to obtain the final RCA products.

(3) Synthesize RCA-AuNPs

The solution of RCA products obtained from Step (2) was mixed with 1 mL of ssDNA-AuNPs solution obtained from Step (3), after which the mixed solution was treated at 95° C. for 10 min, quickly cooled in an ice bath for 1 min, and incubated at 37° C. for 30 min to allow hybridization between RCA products and ssDNA-AuNPs conjugates. After this step, the resulting solution was centrifuged for 20 min at 400-22,000×g (depending on the size of AuNPs), and the supernatant was removed, leaving the ssDNA-AuNPs complex at the bottom. The RCA-AuNPs complex was then resuspended in PBS (0.01 M, pH 7.4). This washing step was repeated for a total of three times.

TABLE 1

| | Se- | |
|---|---|---|
| | quence | |
| Name | # | Sequence (5′ to 3′) |
| Capturing | SEQ ID | NH₂, 12C spacer-CAG |
| aptamer | NO: 1 | CAC CGA CCT TGT GCT |
| (i.e., | | TTG GGA GTG CTG GTC |
| aptamer | | CAA GGG CGT TAA TGG |
| specific | | ACA (51-nt) |
| against | | |
| spike | | |
| protein | | |
| receptor | | |
| binding | | |
| domain | | |
| on the | | |
| surface | | |
| of SARS- | | |
| CoV-2 | | |
| virus) | | |
| RCA | SEQ ID | GGA CAT TTT |
| primer | NO: 2 | TTTTTTTTTTTT CAG CA |
| | | (26-nt) |
| RCA | SEQ ID | Phos-AAA AAA AAT |
| padlock | NO: 3 | GTC CAT TAA CGC CCT |
| probe | | TGG ACC AGC ACT CCC |
| | | AAA GCA CAA GGT CGG |
| | | TGC TGA AAA AAA A-3 |
| | | (67-nt) |
| ssDNA | SEQ ID | Thio, 6Cspacer-AAA AAA |
| | NO: 4 | AAAAAAAAA A |
| | | (16-nt) |

Note:
The underlined portion in the RCA padlock probe sequence (SEQ ID NO: 3) is complementary to the sequence of capturing aptamer in order to produce tandem repeating aptamer (SEQ ID NO: 1) by the RCA reaction. The ssDNA (SEQ ID NO: 4) is complementary to the sequence of the RCA product.

Relevant performance tests of the above embodiment are as follows.

(1) Characterization of Surface Modifications

Fluorescence Labeling Methods

To confirm the success of each surface modification steps, fluorescently labeled molecules (i.e., FITC-G3.5-COOH, rhodamine-G4-NH₂, and cy3-aptamer-NH₂) were employed to substitute their respective molecules to modify each surface layer. Since the layer modified with fluorescently labeled molecules can emit fluorescence signals to indicate the success of surface modifications, the fluorescence intensity changes before and after surface modifications were compared.

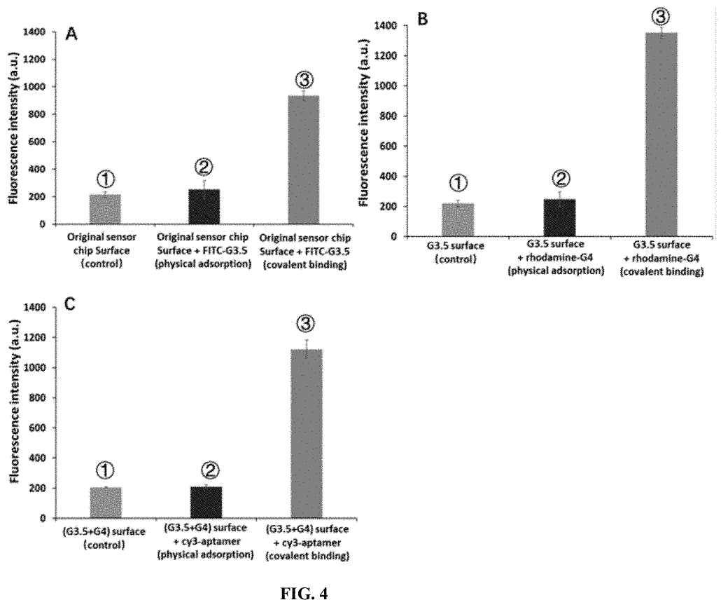

As shown in FIG. 4, compared with the control surfaces (i.e., group \textcircled{1} in A, B, and C), there were significant (p<0.05) increases in fluorescence intensities after the surfaces were modified with fluorescently labeled molecules (see group \textcircled{3} in A, B, and C), indicating the success of each step of surface modifications. Moreover, there was no significant (p>0.05) difference in fluorescence intensities between the control surfaces (i.e., group \textcircled{1} in A, B, and C) and surfaces with physical adsorption of fluorescently labeled molecules (i.e., group \textcircled{2} in A, B, and C), further confirming the success of surface modifications.

Comparison of LSPR Surface Modification Signal

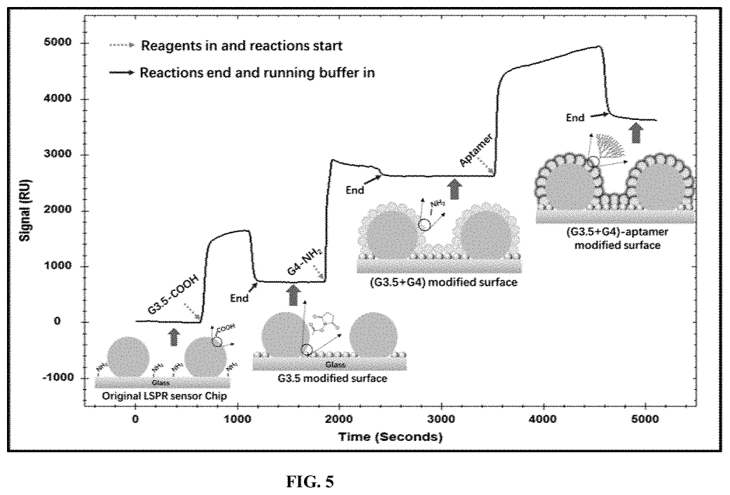

To further demonstrate the success of the surface modification step, the original sensor chip was surface modified in site using a micro fluidic-based LSPR device. In the present embodiment, the LSPR signals of each modification layer were compared. As shown in FIG. 5, LSPR signals were increased after each step of surface modifications, reflecting a surface mass increase of each layer due to the successful immobilization of biomolecules (i.e., G3.5, G4, and aptamers). Specifically, the signal of the original sensor chip surface showed a small value of baseline signal pointing at 10 RU, and significantly (p<0.05) increased to 706 RU after injection of G3.5-COOH immobilization reagents, indicating successful immobilization of G3.5-COOH molecules on the original sensor chip surface. Moreover, after employing G4-NH₂ molecules on the G3.5-COOH modified surface, the signal showed a 4-time increase with a value of 2708 RU, indicating successful surface immobilization of G4-NH₂ molecules. In addition, the surface LSPR signals were further enhanced to 3570 RU after aptamer-conjugation reactants were applied on the (G3.5+G4) modified surface, strongly suggesting that the surface modification with aptamers was successful.

(2) Characterization of ssDNA-AuNPs and RCA Products

ssDNA-AuNPs

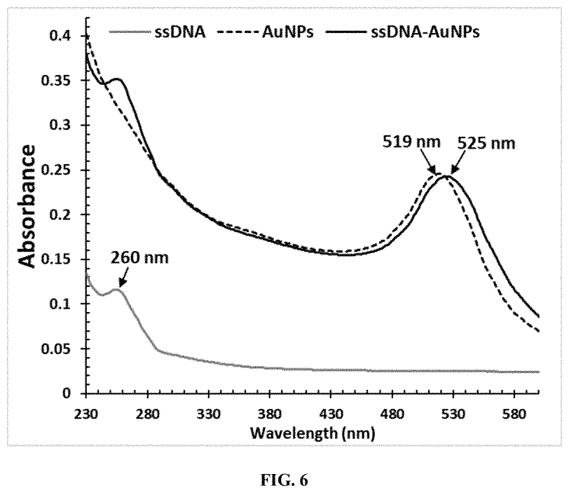

To confirm the successful synthesis of AuNPs-ssDNA conjugate, the samples were analyzed by ultraviolet-visible spectroscopy. As shown in FIG. 6, in comparison with the AuNPs peaked at 519 nm, the AuNPs-ssDNA presented an ssDNA-specific absorbance peak at 260 nm and showed a 6 nm red shift peaked at 525 nm. These observations suggested that the AuNPs were successfully conjugated with ssDNA.

RCA Products

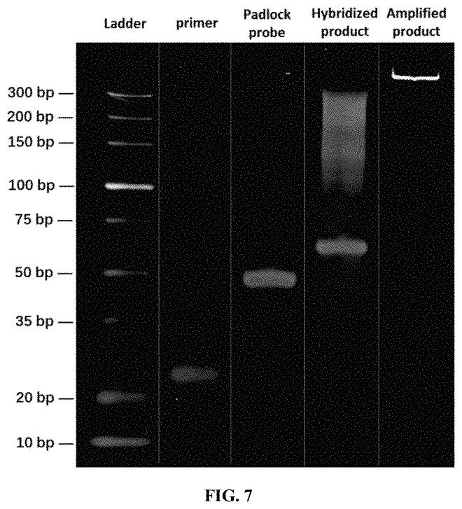

To confirm the successful synthesis of RCA products, polyacrylamide gel (8%) electrophoresis was employed. As shown in FIG. 7, the primer and padlock probe showed distinct bands around 20 and 45 bp, respectively; the band of hybridized products moved to around 65 bp, approximately the total molecular weight of the primer and padlock probe, indicating the success of hybridization. However, the amplified RCA products presented extremely low mobility, as it was trapped in the well of the gel due to high molecule weight, strongly suggesting the success of the RCA reaction.

(3) Performances of (G3.5+G4)-Aptamer Sensor Chips

In the present embodiment, the nonfouling property of the (G3.5+G4)-aptamer sensor chips was compared toother sensor chips with different surface modification configurations by evaluating the detection background noises generated by incubation with 1 mg/mL bovine serum albumin (BSA) as a nonspecific molecule. Specifically, these sensor chips for the nonfouling test were: (1) the original sensor chips functionalized directly with aptamers (i.e., gold-aptamer, curve\textcircled{1}), (2) the sensor chips with G3.5 modification on glass areas in-between the gold nanoislands and functionalized with aptamers (i.e., G3.5-aptamer, curve\textcircled{2}), (3) the sensor chips with G4 modification on the gold nanoislands and functionalized with aptamers (i.e., G4-aptamer, curve\textcircled{3}), and (4) the sensor chips with both G3.5 and G4 modifications and functionalized with aptamers (i.e., (G3.5+G4)-aptamer, curve\textcircled{4}).

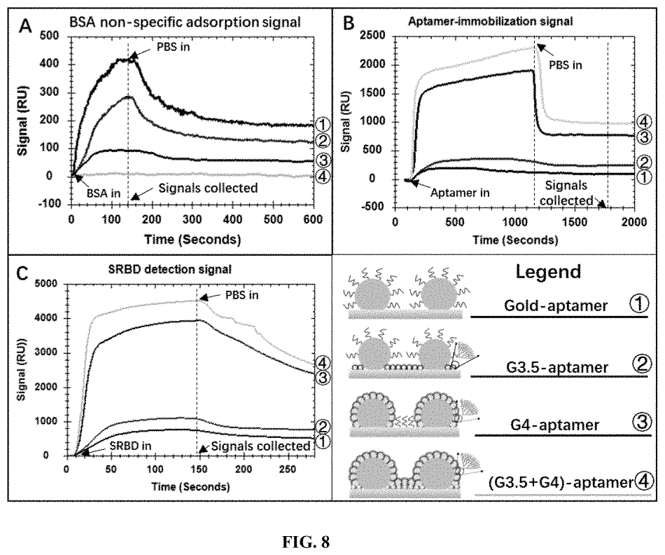

As shown in FIG. 8A, the (G3.5+G4)-aptamer modified sensor chips exhibited the lowest background noises, suggesting excellent nonfouling properties of the modified surfaces; in contrast, gold-aptamer modified sensor chips showed the highest background noises, indicating the highest amount of nonspecific surface adsorption. The G3.5- and G4-aptamer modified sensor chips demonstrated significantly improved nonfouling properties compared to the gold-aptamer modified surfaces, as the background noises of these sensor chips were substantially lower than those of the gold-aptamer modified. This observation suggests that nonspecific adsorption occurs on both gold nanoislands and areas between the nanoislands and that the combined immobilization of G3.5 and G4 molecules can effectively prevent nonspecific adsorptions in both areas. In this embodiment, the sensor chip surface is covered with a layer of dendrimer molecules using a combination of (G3.5+G4) surface modification methods, which results in significantly reduced background noises, thus improving the detection performance of the LSPR sensor chips by enhancing the signal-to-noise ratio. It is worth mentioning that the excellent nonfouling properties of the surface-modified sensor chip eliminate the need for both blocking agents and reference channels specifically designed to reduce and compensate for surface nonspecific bindings in the LSPR assays, respectively.

Moreover, the relative amount of aptamers immobilized on the sensor chip surfaces with different surface modification configurations (i.e., gold-aptamer, G3.5-aptamer, G4-aptamer, and (G3.5+G4)-aptamer surfaces) was studied by directly evaluating the LSPR sensor graph signal changes before and after aptamer immobilizations. As shown in FIG. 8B, the gold-aptamer surface showed the lowest signal, suggesting the lowest amount of immobilized aptamers, due to a limited number of aptamer binding sites on the gold-nanoislands. In contrast, the (G3.5+G4)-aptamer sensor chip surface exhibited the highest aptamer immobilization signal, approximately 10 times higher than that of the gold-aptamer surface. This significant increase in immobilized aptamers amount was caused by the presence of multi-handled G4 dendrimer templates (i.e., 64 binding sites per G4 molecule) via which the multi-copy aptamers could be immobilized on the sensor chip surfaces. This conclusion was further supported by the observation that the immobilized aptamers on the G4-modified sensor chip surface were 4 times higher than the G3.5-modified sensors. Therefore, it can be concluded that the (G3.5+G4) surface-modification configuration allows for the largest amount of aptamers to be tethered on the sensor chip capturing surfaces.

Furthermore, to evaluate the detection performance of the four modified sensor chips (i.e., gold-aptamer, G3.5-aptamer, G4-aptamer, and (G3.5+G4)-aptamer surfaces), these sensor chips were employed to detect SRBD samples. As shown in FIG. 8C, the detection signals from all 4 sensor chips showed patterns and trends similar to that of the surface-immobilized aptamer signals seen in FIG. 8B. Specifically, the (G3.5+G4)-aptamer sensor chip showed the highest detection signal of 4490 RU, while detection signals from other sensor chips were 735 RU (gold-aptamer surface), 1080 RU (G3.5-aptamer surface), and 3900 RU (G4-aptamer surface), respectively. These results strongly indicate that the (G3.5+G4) surface modification on the sensor chip is the most effective design in conjugating a higher density of aptamers to the capturing surface, which results in an improved detection signal in comparison with other sensor chip modification approaches investigated in this study.

To further investigate the detection performances of the (G3.5+G4)-aptamer sensor chips, different concentrations of SRBD samples were tested using both (G3.5+G4)-aptamer and gold-aptamer sensor chips, respectively. As shown in FIG. 9, compared to gold-aptamer sensor chips, (G3.5+G4)-aptamer modified sensor chips showed the highest detection signal at any given SRBD concentration. In addition, it is worth noting that the detection range of the (G3.5+G4)-aptamer modified sensor chip (0.04-377.36 nM) was wider than that of the gold aptamer modified sensor chip (0.19-60.38 nM). This significantly increased detection range exhibited by the (G3.5+G4)-aptamer modified sensor chips was most likely caused by the presence of a greater amount of capturing aptamers on the (G3.5+G4) modified surface than on the non-modified sensor chips, thereby providing more target capturing capacity by allowing both more binding sites for target capturing and stronger binding avidity. Moreover, the slope of the linear region of the response curve for the (G3.5+G4)-aptamer modified sensor chips (k=367.56) was significantly greater (p<0.05) than those for the gold-aptamer (k=48.71), suggesting a much higher detection sensitivity by the (G3.5+G4)-aptamer modified sensor chips, as detection sensitivities are determined by the slope of the response curve. Furthermore, the limit of detection (LOD), defined as the lowest target concentration to provide a signal at least three standard deviations greater than the signal from a negative control, was also calculated for each type of sensor chip. The results showed that the LOD of (G3.5+G4)-aptamer modified sensor chip is 21.9 pM, which is about 9 times more sensitive than that of gold-aptamer modified sensor chip (205.2 pM).

(4) SARS-CoV-2 Virus Detection and Signal Amplification

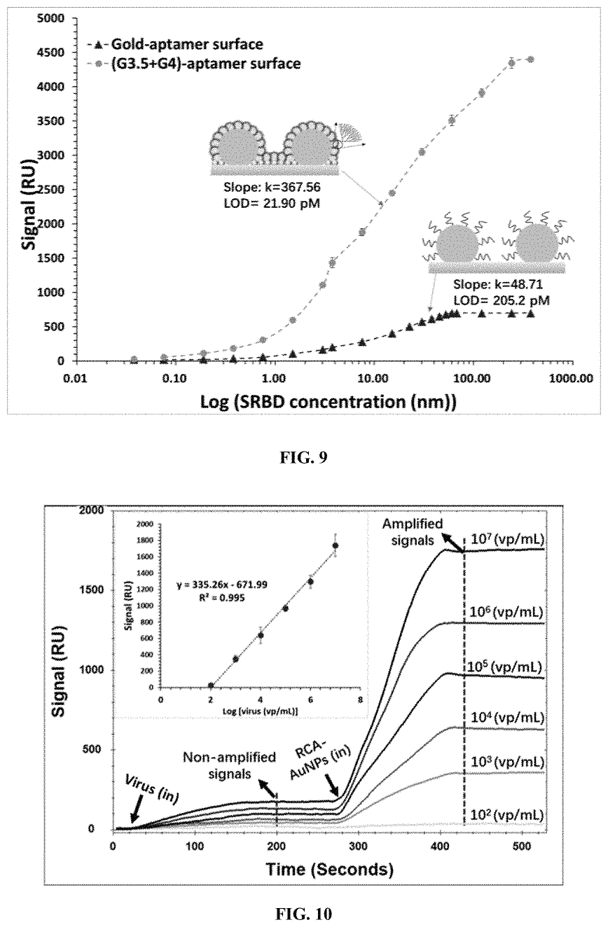

To evaluate the detection performances of the SARS-CoV-2 virus with RCA-AuNPs signal amplification, different concentrations of SARS-CoV-2 virus were detected using the (G3.5+G4)-aptamer modified sensor chips, followed by a signal amplification using RCA-AuNPs. As shown in FIG. 10, the sensor graph presented low detection signals at any virus concentration before signal amplification was employed; in contrast, the detection signals were increased approximately 10-fold after the RCA-AuNPs complex was applied, indicating successful (and significant) detection signal intensification. Moreover, the LSPR signal was proportional to the logarithmic value of the virus concentration with a linear equation of y=335.26x−671.99 (R²=0.995) (inset), and the LOD was calculated to be 148 viral particles per milliliter (vp/mL), one of the best sensitivities reported in whole viral particle detections amongst all detection platforms. It should be noted that the typical SARS-CoV-2 viral concentration from nasopharyngeal and saliva swabbed samples is 10⁴-10¹⁰ vp/m, suggesting that the currently reported sensor chip modification and signal amplification approach can be used for early infection diagnostics to sensitively detect the SARS-CoV-2 virus. Moreover, the current approach directly detects whole viral particles; therefore, no sample pre-treatment (e.g., protein or gene extraction) would be required, and the whole detection time can be done in less than 3 min, more efficient than any existing methods that require laborious sample preparations. In addition, the modified sensor chip can be regenerated multiple times using a regeneration solution (i.e., glycine-HCL (10 mM, pH 2.0)), significantly saving the overall detection time and cost.

(5) Detection Specificity and Influence of Biological Mixtures

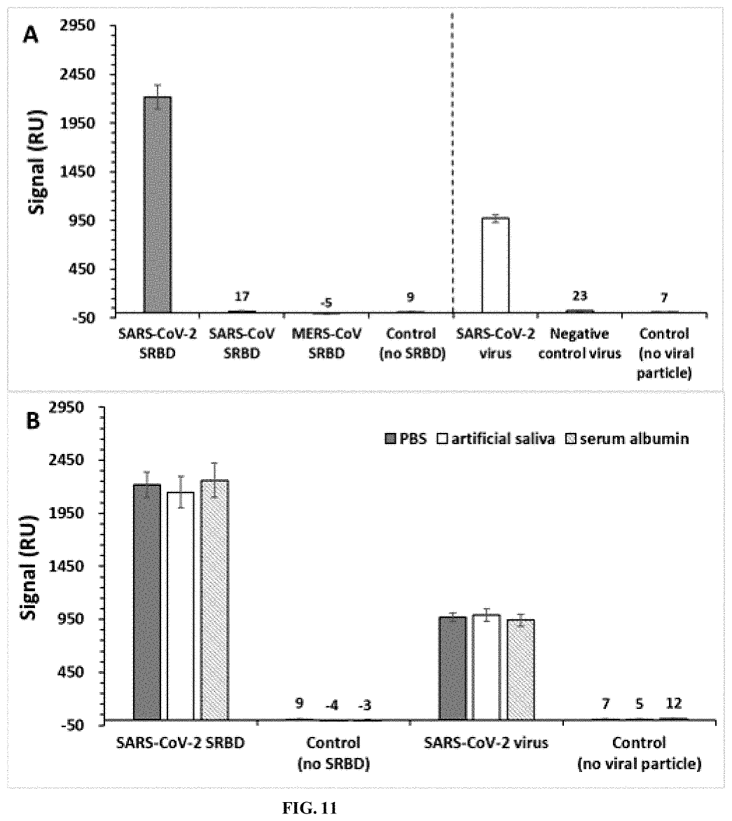

To study the specificity of (G3.5+G4)-aptamer sensor chip for detecting the SRBD samples, the target (i.e., SARS-CoV-2 SRBD) and non-targets (i.e., SARS-CoV SRBD and Middle East Respiratory Syndrome (MERS)-CoV SRBD) were tested. As shown in FIG. 11A (gray bars), signals for the target (i.e., SARS-CoV-2 SRBD) were significantly higher than those of non-targets (i.e., SARS-CoV SRBD and MERS-CoV SRBD), suggesting excellent detection specificity of the sensor chip for SARS-CoV-2 SRBD. To further study the specificity for viral particles detections, the SARS-CoV-2 virus (i.e., targets) and negative control virus with no spike protein on the surface (i.e., non-targets) were detected using the (G3.5+G4)-aptamer sensor chip followed by signal amplification with RCA-AuNPs.

As shown in FIG. 11A (white bars), the negative control virus (i.e., non-target) showed a low signal value at 23 RU compared to the SARS-CoV-2 virus (i.e., target) with a signal value at 970 RU, indicating excellent detection specificity for SARS-CoV-2 viral particles. Moreover, the weak signal (7 RU) of the control sample (i.e., no viral particles) indicates minimal nonspecific interactions between the detection surface and the RCA-AuNPs.

To investigate the (G3.5+G4)-aptamer sensor chip performance under conditions that mimic real-world conditions, SARS-CoV-2 SRBD and SARS-CoV-2 virus were analyzed in PBS (pH 7.4), artificial saliva (1% v/v), and BSA (40 μg/mL) solutions, as biological matrices are known to influence detection performance. As shown in FIG. 11B, in comparison with signals analyzed under the PBS (pH 7.4) condition, there was no significant (p>0.05) difference in signals when either SRBD or SARS-CoV-2 virus samples were analyzed in saliva and BSA matrices. This is due to the excellent non-fouling surface property of the detection surface, suggesting that the modified sensor chips were sufficiently robust to detect samples in complex biomatrices.

While the invention has been described with reference to specific embodiments, the invention is not limited thereto, and various equivalent modifications and substitutions can be easily made by those skilled in the art within the technical scope of the invention. Therefore, the protection scope of the present invention shall be subject to the protection scope of the claims.

1. A biosensor chip that comprises a sensor chip body, wherein the detection surface of the sensor chip body is featured with a template layer; the template layer has multiple binding sites that bind to the detection ligands.

2. The biosensor sensor chip of claim 1, wherein the template layer is constructed by immobilizing functional polymers on the detection surface of the sensor chip body; wherein the detection ligands are aptamers, antibodies, peptides, receptors, polymers, or enzymes.

3. The biosensor sensor chip of claim 2, wherein the functional polymers comprise one or more types of polymers, such as dendritic, linear, and crosslinked polymers.

4. The biosensor sensor chip of claim 3, wherein the functional polymer is PAMAM dendrimer; the PAMAM dendrimer comprises at least one of Generation 3.5 carboxylated PAMAM dendrimers and the Generation 4 aminated PAMAM dendrimers.

5. A COVID-19 test kit, wherein comprises a biosensor chip as described in any of the claims 1-4, rolling circle amplification primers, i.e., RCA primers; RCA padlock probes; and ssDNA conjugated nanoparticles, i.e., ssDNA-NPs probes.

6. The COVID-19 test kit of claim 5, wherein the nanoparticles, i.e., NPs comprise at least one of gold NPs, silver NPs, platinum NPs, copper NPs, and non-metallic NPs with metal coatings.

7. The COVID-19 test kit of claim 5, wherein the sequence of ssDNA is SEQ ID NO:4.

8. The COVID-19 test kit of claim 5, wherein the sequence of RCA primers is SEQ ID NO:2.

9. The COVID-19 test kit of claim 5, wherein the sequence of RCA padlock probes is SEQ ID NO:3.
